(19) United States
(12) Patent Application Publication
(10) Pub. No.: US 2007/0074991 A1
(43) Pub. Date: Apr. 5, 2007

(54) Ear plug package and method of manufacture

(76) Inventor: David M. Heisserer, Minneapolis, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/542,622

(22) Filed: Oct. 3, 2006

Related U.S. Application Data
(60) Provisional application No. 60/723,240, filed on Oct. 3, 2005.

(51) Int. Cl. A61B 19/02; B65D 85/00; B65D 83/04; A61F 11/00

(52) U.S. Cl. 206/438; 128/864; 206/526; 206/540

(57) ABSTRACT

An ear plug package is provided to compressibly and compactly store one or more earplugs. The ear plug package comprises a base, a body, and a retaining seal. The base is coupled to the body, and the body includes one or more compartments, each adapted to receive and store at least one compressible earplug. The retaining seal is removable, and may comprise multiple sections, with perforations between sections. The ear plug package also may include one or more surfaces ideal for printing advertising, logos, images, or the like. In a preferred embodiment, the base includes at least one print surface. In another embodiment, the seal is provided with a print surface, in addition to or in place of a print surface on the base. The ear plug package is dimensionally similar to a credit card or business card.

The present application claims the benefit of U.S. Provisional Application No. 60/723,240 filed Oct. 3, 2005, which is incorporated herein in its entirety by reference.

### FIELD OF THE INVENTION

The present invention relates generally to packaging for consumer products. More specifically, the present invention is packaging suitable for holding ear plugs.

### BACKGROUND OF THE INVENTION

It is desirable or may be required by law to provide hearing protection for persons exposed to environments where noises are generated sufficiently loud to constitute a danger to hearing. Hearing protection is important in the workplace, especially for those employed at factories, construction sites, airports, and so forth. Hearing protection is equally important in leisure and recreation activities, such as music concerts or motor sport events. One form of hearing protection is earplugs, the use of which is well known, with many varieties of earplugs offered to consumers.

Foam earplugs are generally well accepted for providing hearing protection in the form of sound attenuation. Such earplugs are generally cylindrical in shape and of somewhat larger diameter than the human ear canal. The earplugs may be composed of a foamed plasticized polymeric material having a sufficiently high concentration of plasticizer to provide the earplug with a reduced rate of recovery from compression. The earplugs can be compressed by rolling them lengthwise between one's fingers. The compressed earplug is inserted into the ear canal where it then recovers slowly until it conforms to and obdurates the ear canal, thus suppressing or attenuating the transmission of noise.

Many earplugs are designed to have a short lifespan to keep production costs low, and may even be considered disposable after a single use. Earplugs may be packaged in bulk, for example for use on a factory floor. Earplugs may be packaged in individual pairs, suitable for individual resale, or packaged in a small storage container. Additionally, earplug packaging may incorporate usage instructions, warnings, advertising, logos, images, or other indicia printed on the package.

### SUMMARY OF THE INVENTION

The present invention comprises an ear plug package adapted to compressibly and compactly store one or more earplugs. In one embodiment, the ear plug package comprises a base, a body, and a retaining seal. The base is coupled to the body, and the body includes one or more compartments each adapted to receive and store at least one compressible earplug. The retaining seal is removable, and may comprise multiple sections, with perforations between sections. The ear plug package also may include one or more surfaces ideal for printing advertising, logos, images, or the like. In a preferred embodiment, the base includes at least one print surface. In another embodiment, the seal is provided with a print surface, in addition to or in place of a print surface on the base. The ear plug package is dimensionally similar to a credit card or business card.

The ear plug package stores earplugs in a compressed and partially flattened shape. Earplugs are compressed underneath the seal, and a vacuum may or may not be introduced into the compartments. To remove one or more earplugs from the package, the seal is peeled away from the body, the seal separating into a section along a perforation, and the earplugs are removed.

In one embodiment, the retaining seal is designed to be single-use, such that once the retaining seal is lifted from the body, the earplugs cannot be replaced into the compartment from which they originated. In another embodiment, the retaining seal is configured to be re-sealable so that earplugs can be stored in a compartment after use.

In one embodiment, the package comprises a seal coupled directly to a base to compressibly hold earplugs. The base may be rigid or pliable, and the use of a vacuum under the seal may be used to increase the compression of the earplugs.

An advantage of the present invention is the small, convenient size of the ear plug package, having a profile similar to a credit card and easily stored in a pocket, purse, or wallet.

A further advantage of the present invention is the potential for its use as a point-of-sale item. A still further advantage of the invention is its use as a marketing, advertising or promotional aid, or memorabilia device.

### BRIEF DESCRIPTION OF THE DRAWINGS

The present invention can be more completely understood and appreciated by referring to the following more detailed description of the presently preferred exemplary embodiments of the invention in conjunction with the accompanying drawings, of which:

FIG. 1 is a perspective view of an earplug package according to the present invention.

FIG. 2 is a perspective view of the opposite side of the earplug package depicted in FIG. 1.

FIG. 3A is an exploded perspective view of an earplug package according to the present invention.

FIG. 3B is a further exploded perspective view of an earplug package according to the present invention.

FIG. 4 is a still further exploded perspective view of an earplug package according to the present invention.

FIGS. 5A-5D are perspective views of the steps of removing earplugs from an earplug package according to the present invention.

FIG. 6A is a side cutaway view of an earplug package according to the present invention.

FIG. 6B is an exploded view of area B in FIG. 6A.

FIG. 7A is an exploded view of a further embodiment of an earplug package according to the present invention.

FIG. 7B is a perspective view of an earplug package embodiment of FIG. 7A.

FIG. 8 is a perspective view of a further embodiment of an earplug package according to the present invention.

FIG. 9 is a perspective view of a further embodiment of an earplug package according to the present invention.

FIG. 10 is a perspective view of a further embodiment of an earplug package according to the present invention.

### DETAILED DESCRIPTION OF THE DRAWINGS

In the following detailed description of the present invention, numerous specific details are set forth in order to provide a thorough understanding of the present invention. However, it will be obvious to one skilled in the art that the present invention may be practiced without these specific details. In other instances, well-known methods, procedures, and components have not been described in detail so as to not unnecessarily obscure aspects of the present invention.

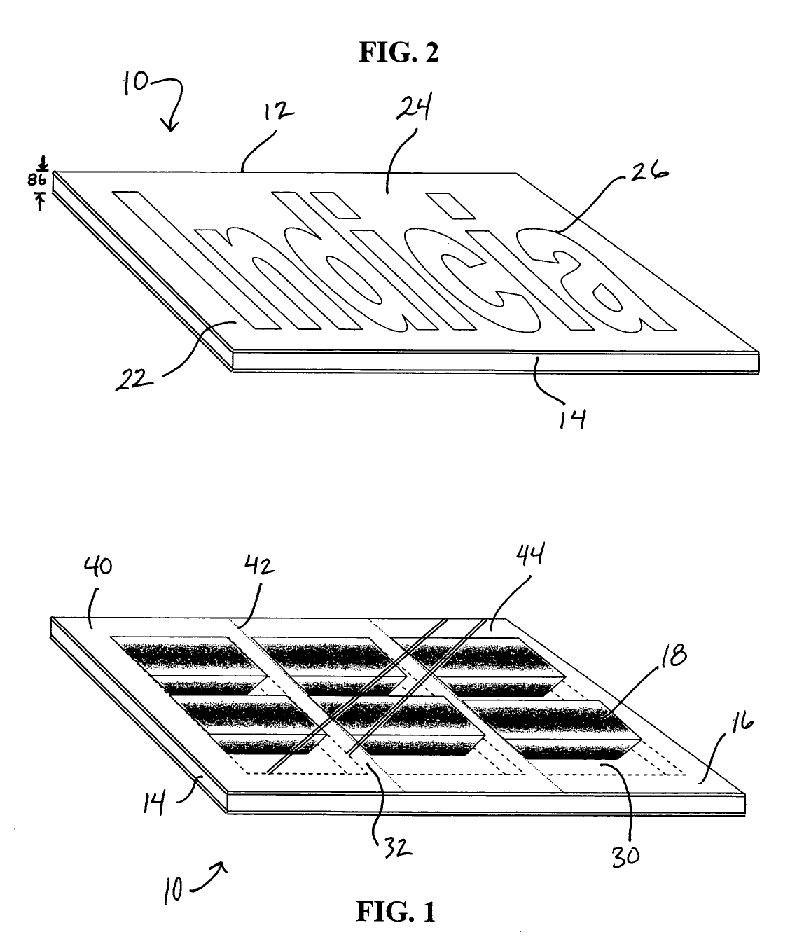

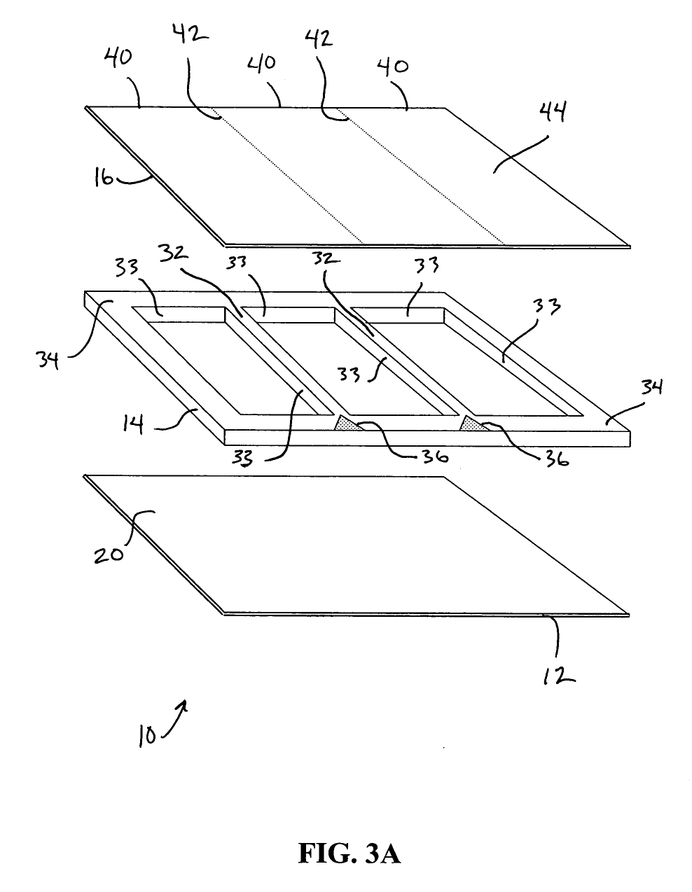

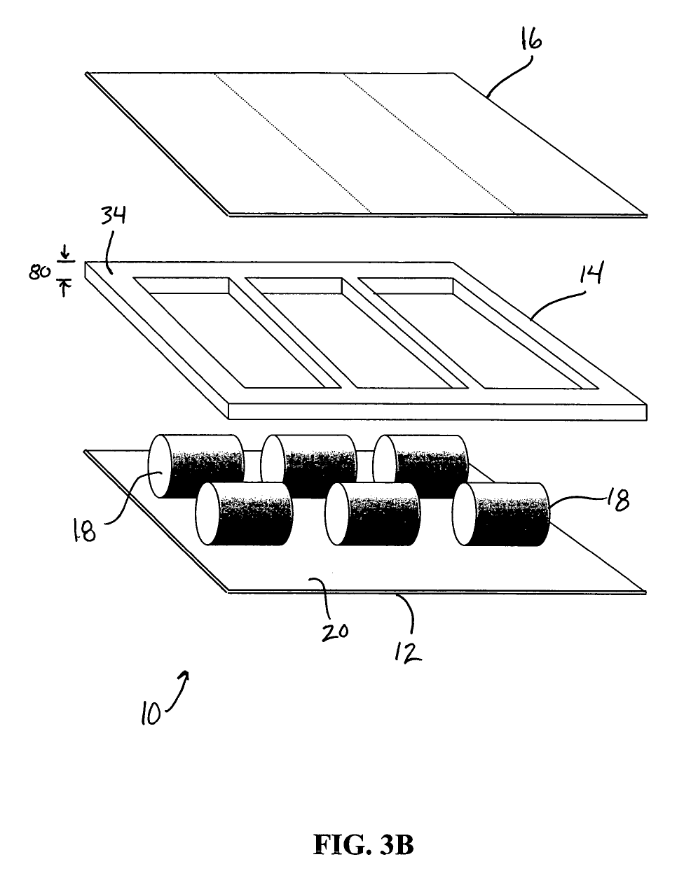

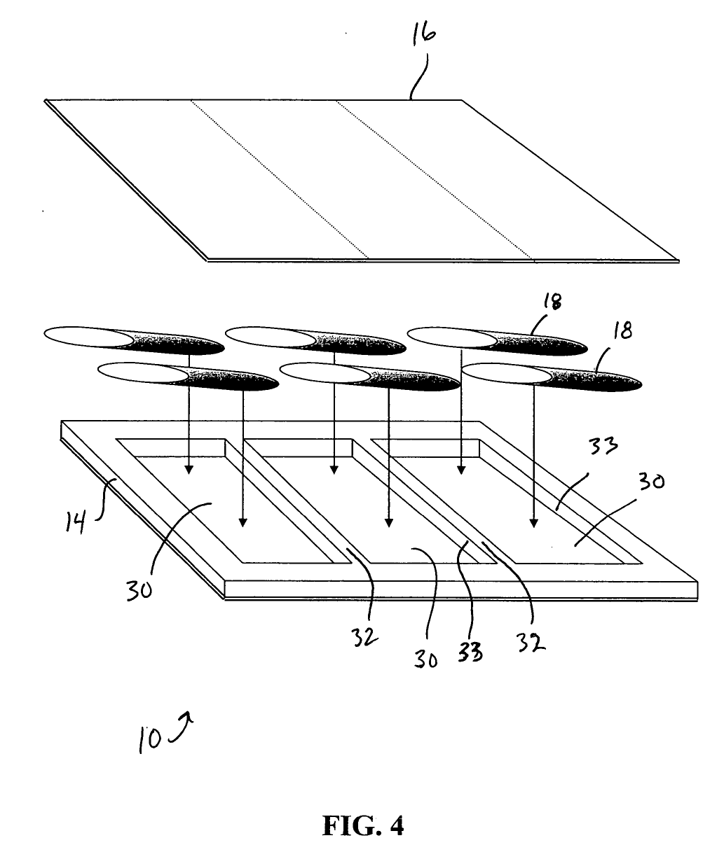

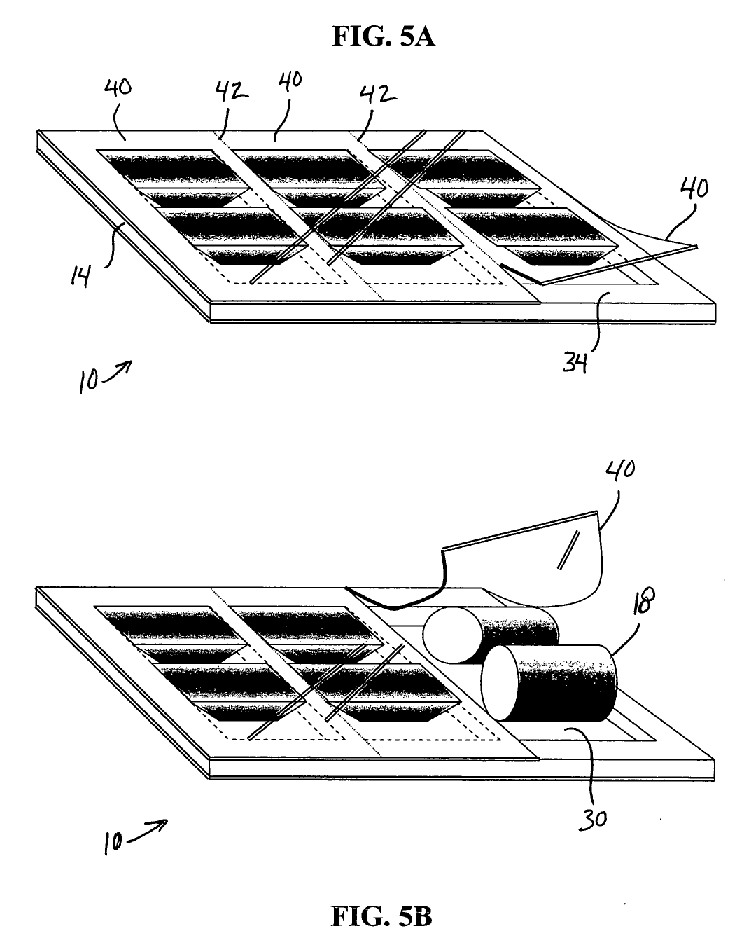

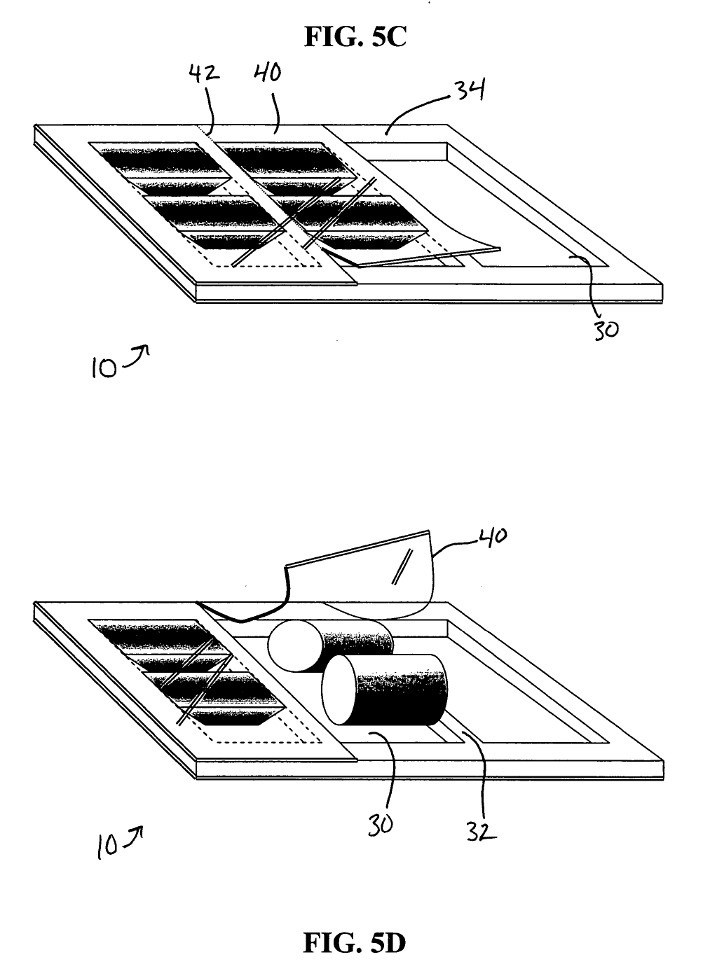

Referring to FIGS. 1-5D, an ear plug package 10 is depicted. Ear plug package 10 includes a base 12, body 14, and a retaining seal 16. Ear plug package 10 is configured to releasably retain one or more compressible earplugs 18, earplugs 18 being of the urethane foam, PVC foam, or silicone rubber type. Preferably, ear plug package 10 is shaped and sized similar to a credit card or business card, albeit slightly thicker to provide for storage of compressed earplugs 18 therein. Ear plug package 10 preferably holds multiple pairs of earplugs 18, however the present invention is not limited to a specific number of earplugs or earplug pairs. In one embodiment, ear plug package 10 is credit card sized, as that term is defined by the standards in ISO/IEC 7810:2003 for an ID-1 class identification card, namely 85.6 mm x 53.98 mm. In such an embodiment, the thickness of ear plug package 10 may be larger than the thickness specified by ISO/IEC 7810:2003, namely 0.76 mm, but the thickness of the package will be 8.0 mm or less.

Base 12 comprises a first side 20 and a second side 22. First side 20 faces the interior of ear plug package 10, while second side 22 is on the exterior of ear plug package 10. Second side 22 defines a print surface 24, suitable for printing, affixing, or otherwise displaying indicia 26 thereupon, as depicted in FIG. 2. Indicia 26 may comprise instructions, warnings, advertising, images, logos, marks, artwork, affiliations, promotions, or other designs. Base 12 is preferably sturdy, durable, and inexpensive, yet somewhat pliable. Ideal materials for the construction of base 12 include cardboard, paperboard, thermoplastics such as polypropylene or polyethylene, or other similar materials as will be apparent to one skilled in the art. Base 12 is preferably substantially planar and continuous, so as to provide maximum surface area for displaying indicia 26. In one embodiment, base 12 may be separable from body 14 such that after a user has used all the earplugs 18 within package 10, base 12 may be removed and retained by the user, thereby retaining the indicia printed on base 12.

Body 14 includes one or more compartments 30, defining a space adapted to receive and store one or more earplugs 18. If body 14 includes more than one compartment 30, a partition or divider 32 separates each adjacent compartment. Compartment 30 may contain one or more earplugs 18. Each compartment includes a closed bottom, defined by base 12, and a plurality of inner sides 33 defined by body 14 itself, or dividers 32, or a combination thereof. As depicted in FIG. 1, for example, each compartment 30 contains two earplugs 30, however ear plug package 10 may be configured such that an individual earplug 18 is stored in a compartment 30, or ear plug package 10 may be configured such that multiple earplugs are stored in a single compartment 30, without any partitions 32. Body 14 also includes a sealing surface 34 adapted to releasably receive seal 16, and sealing surface 34 may feature one or more recesses 36 configured to ease and facilitate removal of seal 16. Ideal materials for the construction of body 14 include cardboard, paperboard, thermoplastics such as polypropylene or polyethylene, or other similar materials as will be apparent to one skilled in the art. Body 14 and base 12 are non-releasably coupled, such as by adhesive, heat, or chemical fusion. In an alternate embodiment, body 14 and base 12 may comprise a unitary element.

Retaining seal 16 is releasably coupled to sealing surface 34, and is configured to retain one or more earplugs 18 in compartments 30 until such time as is desired to remove the earplugs from package 10. Seal 16 is preferably coupled to sealing surface 34 of body 14 by means of adhesion, wherein a suitable adhesive should adequately hold seal 16, yet easily allow a user to peel away seal 16. If body 14 is divided into multiple compartments 30, seal 16 is divided into multiple peelable sections 40, or peels, corresponding to the compartments such that removing a section 40 exposes a compartment 30 beneath it. Perforations 42 are included between sections 40, such that a section 40 of seal 16 is easily removable to obtain earplugs 18 from underneath the section. Seal 16 may optionally include a print surface 44, similar to print surface 24 on base 12. Print surface 44 may be on the exterior of seal 16, or on the interior of seal 16 in an embodiment wherein seal 16 is transparent. Print surface 44 is suitable for printing, affixing, or otherwise displaying indicia thereupon, such as instructions, warnings, advertising, images, logos, marks, artwork, affiliations, promotions, or other designs. Seal 16 may be transparent, translucent, or opaque. Suitable materials for seal 16 include thermoplastics such as polypropylene or polyethylene, paper, foil, a combination thereof, or other materials apparent to one skilled in the art. Seal 16 may comprise a single layer of material, or multiple layers laminated or otherwise secured together.

In an embodiment where earplugs 18 are stored in compartments 30 under negative pressure, compartments 30 are configured to withstand the vacuum. Base 12, body 14 and seal 16 will each be configured to include an air impenetrable layer. Adhesives used to secure base 12 to body 14 and seal 16 to body 14 are similarly configured to withstand pressure less than standard atmospheric pressure within compartments 30. The use of a suitable chemical conditioner, lubricant, or similar on earplugs 18 may be necessary to more effectively package earplugs 18 within a vacuum, as will be apparent to one skilled in the art.

Seal 16 may be single-use such that after opening it is not re-sealable, or reusable to facilitate repeated opening and closing. If seal 16 is configured to be reusable, an appropriate re- sealable adhesive is utilized between body 14 and seal 16.

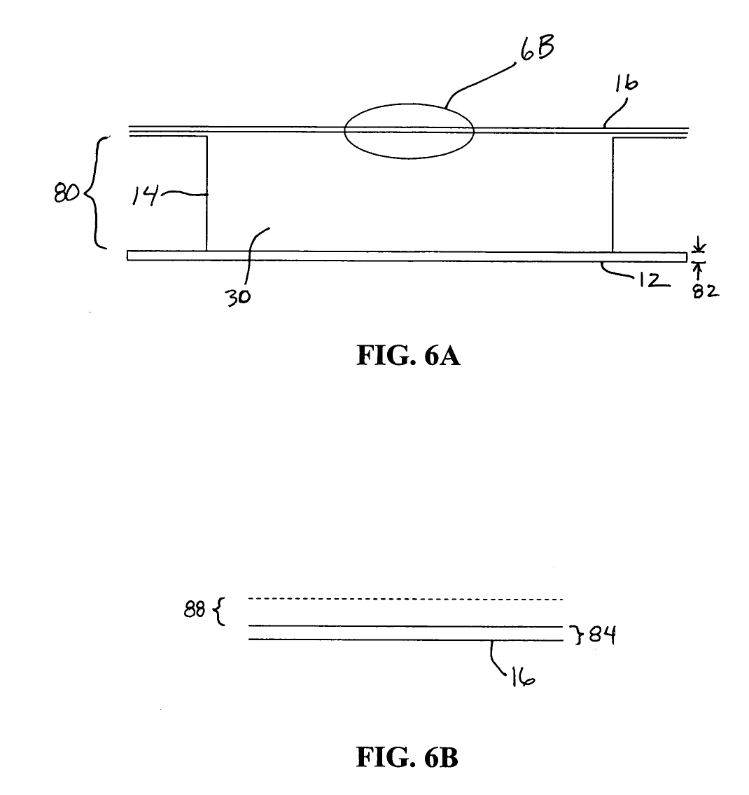

Body 14 preferably has a thickness 80 between 1.0 mm and 4.0 mm, and base 12 preferably has a thickness 82 between 0.5 mm and 2.0mm, as depicted in FIG. 6A. The thickness of seal 16 is dependent on the material used for seal 16, the amount of compression of earplugs 18, the material of earplugs 18, and the pressure within compartment 30. Preferably, the thickness of seal 16 is between 0.5 mil and 7 mil. The overall thickness 86 of package 10 is preferably 8.0 mm or less. Package 10 may also have an overall thickness 86 of 7.0 mm or less, 6.0 mm or less, 5.0 mm or less, 4.0 mm or less, 3.0 mm or less, or 2.0 mm or less.

Preferably, earplugs 18 are compressed into a flattened rectangular shape such as is depicted in FIG. 1. Earplugs 18 are compressed to have a height of 6 mm or less when stored in compartment 30. However, earplugs 18 may be compressed to a height of 5.0 mm or less, 4.0 mm or less, 3.0 mm or less, 2.0 mm or less, or a height of 1.0 mm or less. Alternatively, earplugs 18 may be compressed into a cylindrical shape. The deflection 88 of seal 16 when earplugs 18 are placed in compartment 30 is preferably 2.0 mm or less and ideally 1.0 mm or less, as depicted in FIG. 6B. Deflection 88 may be in a direction away from earplug 18, or toward earplug 18, in the case of negative pressure within compartment 30. The type of material used and the thickness of seal 16 should be selected so as to provide a deflection of 2.0 mm or less.

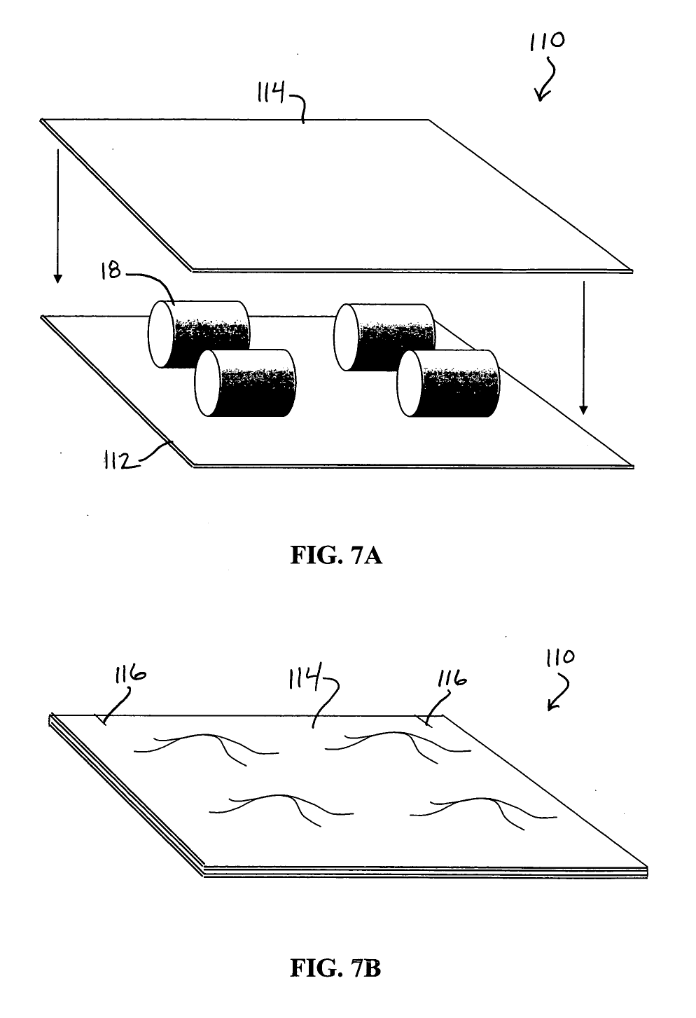

A number of variations on the above mentioned ear plug package 10 are within the scope and spirit of the present invention. Referring now to FIGS. 7A and 7B, an ear plug package 110 is depicted. Package 110 includes a base 112 and a seal 114 affixed directly to base 112. Package 110 is similar to ear plug package 10, however package 110 does not include a body between the base and the seal. Rather, earplugs 18 are retained between base 112 and seal 114. Base 112 is similar to base 12 in earlier embodiments, and includes an outwardly facing print surface suitable for printing, affixing, or otherwise displaying indicia thereupon. The indicia may comprise instructions, warnings, advertising, images, logos, marks, artwork, affiliations, promotions, or other designs. In one embodiment, base 112 is sturdy and durable, yet somewhat pliable, and constructed from cardboard, paperboard, or the like. Base 112 is preferably substantially planar and continuous, so as to provide maximum surface area for displaying indicia. Seal 114 is affixed directly to base 112, retaining compressed earplugs 18 therein. A vacuum may be applied under seal 114 during the manufacturing process to reduce the overall thickness of ear plug package 110. Seal 114 is similar to seal 16 discussed in earlier embodiments, in that seal 114 is configured to retain one or more earplugs 18 until such time as is desired to remove the earplugs. Seal 114 is preferably coupled to body 112 by means of adhesion, and seal 114 may include perforations to divide seal 114 into multiple sections. Seal 114 may also include a print surface for printing, affixing, or otherwise displaying indicia thereupon. Seal 114 may be transparent, translucent, or opaque, and may comprise a single layer of material or multiple layers laminated or otherwise secured together. Suitable materials for seal 114 include thermoplastics such as polypropylene or polyethylene, paper, foil, a combination thereof, or other materials apparent to one skilled in the art. Pre-cut slits 116 may be included in package 110 for ease of removal of seal 114.

In another embodiment of ear plug package 110, base 112 is not rigid, but rather is extremely pliable, flexible, and compliant. Base 112 is preferably constructed from a thermoplastic such as polypropylene or polyethylene, or foil, a combination thereof, or other similar materials as will be apparent to one skilled in the art. When coupled with seal 114, ear plug package 110 is flexible and conformable, and is easily stored in a pocket, wallet, purse, or other convenient location. The use of a vacuum may be incorporated during the manufacturing process

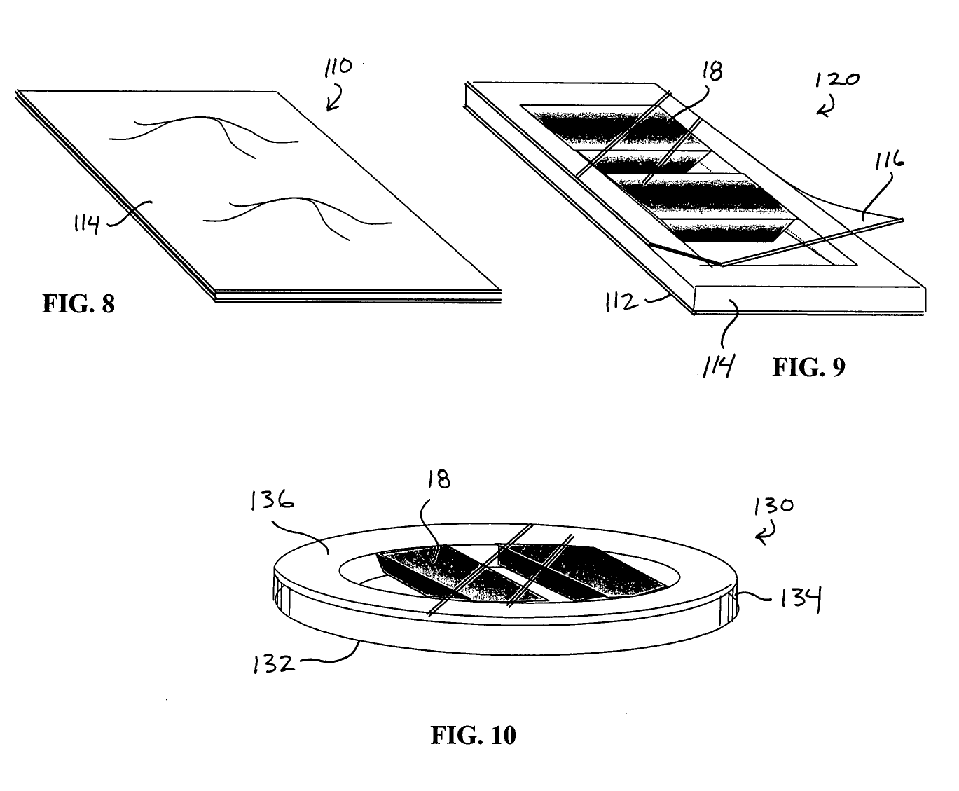

Referring now to FIG. 8, a further embodiment of ear plug package 110 is depicted, containing only two earplugs 18. In this embodiment, ear plug package is smaller in size than the embodiments in FIGS. 7A and 7B, and is similar in size to a ketchup packet. Package 110 is depicted as holding two earplugs, and depicted in FIGS. 7A and 7B as holding four earplugs, however this should not be considered limiting.

Referring now to FIG. 9, an ear plug package 120 is depicted. Package 120 is substantially similar to ear plug package 10 depicted in FIGS. 1-5C, although smaller in size to accommodate only two earplugs 18. The structure and function of ear plug package 120 is the same as that of ear plug package 10, and includes a base 122, a body 114, and a seal 116. Ear plug package 120 is similar in size to a ketchup packet.

A further embodiment of the present invention is depicted in FIG. 10. Ear plug package 130 is substantially similar to packages 10 and 120, discussed previously. The structure and function of ear plug package 130 is substantially the same as that of ear plug package 10, including a base 132, a body 134, and a seal 136. Ear plug package 130 comprises a circular profile, which may be more desirable to prospective purchasers, and may be more easily stored and transported.

Although the present invention has been described with reference to particular embodiments, one skilled in the art will recognize that changes may be made in form and detail without departing from the spirit and scope of the invention. Therefore, the illustrated embodiments should be considered in all respects as illustrative and not restrictive.

1. A package for ear plugs, comprising:
a body having a base, a top defining a sealing surface, and at least one compartment to store one or more compressed ear plugs therein;
a seal releasably secured to the sealing surface of the body and adapted to retain one or more compressed ear plugs under the seal; and
at least one surface suitable for displaying indicia thereupon.

2. The package of claim 1, wherein at least one surface is on the body.

3. The package of claim 1, wherein at least one surface is on the seal.

4. The package of claim 1, wherein the body comprises two or more compartments and a divider between adjacent compartments.

5. The package of claim 4, wherein the seal comprises at least two sections separated by perforations, the sections corresponding to the compartments.

6. The package of claim 1, further including a recess on the body to facilitate removal of the seal.

7. The package of claim 1, wherein the package has a thickness of 8 millimeters or less.

8. The package of claim 1, wherein the package is credit card sized.

9. An earplug package comprising:
a base, having a surface suitable for displaying indicia thereupon;
a sealing surface; and
a seal releasably coupled to the sealing surface and adapted to retain one or more compressed ear plugs under the seal.

10. The earplug package of claim 9, wherein the air pressure under the seal is less than standard atmospheric pressure.

11. The earplug package of claim 9, wherein the seal includes a surface suitable for displaying indicia thereupon.

12. The earplug package of claim 9, wherein the seal is divided into two or more sections.

13. The earplug package of claim 9, wherein the package has a thickness of 6 millimeters or less.

14. A method of manufacturing a package for ear plugs, comprising:
providing a body having a base, a sealing surface, and at least one compartment to house one or more ear plugs;
compressing one or more ear plugs into the at least one compartment;
securing a removable seal to the sealing surface on the body so as to cover the compartment and retain the one or more earplugs therein; and
providing a surface on the package suitable for printing indicia thereupon.

15. The method of claim 14, further comprising creating a vacuum within the at least one compartment.

16. The method of claim 14, wherein the body comprises two or more compartments and a divider between adjacent compartments.

17. The method of claim 16, wherein the seal includes two or more sections separated by perforations corresponding to the compartments.

18. The method of claim 14, wherein the base is separable from the body.

19. The method of claim 14, wherein compressing one or more earplugs comprises compressing one or more earplugs to a height of 6 millimeters or less.

20. The method of claim 14, further comprising making the package credit card sized.
